(19) United States
(12) Patent Application Publication
(10) Pub. No.: US 2010/0221368 A1
(43) Pub. Date: Sep. 2, 2010

(54) Methods for Inhibiting Cytokines

(75) Inventor: Gregory J. Tsay, Taichung City (TW)

Correspondence Address: WPAT, PC;INTELLECTUAL PROPERTY ATTORNEYS, 2030 MAIN STREET, SUITE 1300, IRVINE, CA, 92614, US

(73) Assignee: CHUNG SHAN MEDICAL UNIVERSITY, Taichung City (TW)

(21) Appl. No.: 12/394,878

(22) Filed: Feb. 27, 2009

(51) Int. Cl. A61K 36/48; A61P 19/02

(52) U.S. Cl. 424/757

(57) ABSTRACT

A method for inhibition a cytokine, transgluaminase type 2 (TG2) or matrix metalloproteinase 9 (MMP-9) was provided.

### FIELD OF THE INVENTION

The present invention relates to a method for inhibiting cytokines including interleukin-1β (IL-1β), interleukin-6 (IL-6), transgluaminase type 2 (TG2) or matrix metalloproteinase 9 (MMP-9).

### BACKGROUND OF THE INVENTION

In Chinese literature search, “I-Tiao-Gung” is a common name used in different medicinal plants. For example, the local name of Glycine tomentella Hayata and Moghania philippinensis are “I-Tiao-Gung” because both of them are belong to leguminosae family. The “I-Tiao-Gung” used herein is Glycine tomentella Hayata.

I-Tiao-Gung has been used as a traditional herbal medicine to treat rheumatic diseases in Kinmen and Taiwan, indicative of its anti-inflammatory effect. I-Tiao-Gung has also been showed anti-atherosclerosis and anti-oxidative activities.

Rheumatoid arthritis (RA) is a chronic, systemic autoimmune disorder, the cause for which is still unknown. It causes systemic joints inflammation and may damage some organs. Cytokines give rise to inflammation of joint synovium, such as tumor necrosis factor alpha (TNF-α), interleukins (IL-1, IL-6, IL-8 and IL-15), transforming growth factor beta (TGF-β), fibroblast growth factor (FGF) and platelet-derived growth factor (PDGF). There is no known cure for RA, but many different treatments can alleviate symptoms.

### SUMMARY OF THE INVENTION

The present invention provides a method for inhibiting cytokine expression of a patient comprising administering the patient an effective amount of organic extract from Glycine tomentella Hayata, wherein the cytokine is selected from the group consisting of interleukin-1β (IL-1β), interleukin-6 (IL-6), transgluaminase type 2 (TG2) and matrix metalloproteinase 9 (MMP-9).

### BRIEF DESCRIPTION OF THE DRAWINGS

FIG. 1: Cell viability was measured by MTT and expressed as the mean percentage SEM (n=3) of those control cells (100%). *p<0.05 and **p<0.01 represent significant differences compared with the values seen in control cells.

FIG. 2: Effects of the ethanol extract of I-Tiao-Gung on IL-1β, IL-6 and TG2 mRNA expression in Raw 264.7 cells. (A) The RT-PCR showed IL-1β, IL-6, TG2 stimulated by LPS inhibited by I-Tiao-Gung; (B) IL-1β was inhibited by the ethanol extract of I-Tiao-Gung in a dose-dependent manner; (C) IL-6 was inhibited by the ethanol extract of I-Tiao-Gung in a dose-dependent manner; (D) TG-2 was inhibited by the ethanol extract of I-Tiao-Gung in a dose-dependent manner. (*p<0.05; **p<0.01 as compared with group treated with LPS).

FIG. 3: I-Tiao-Gung reduced MMP-9 secretion compared with cells stimulated only by LPS. (A) The RT-PCR showed MMP-9 stimulated by LPS inhibited by I-Tiao-Gung; (B) MMP-9 was inhibited by the ethanol extract of I-Tiao-Gung in a dose-dependent manner. (*p<0.05; **p<0.01 as compared with group treated with LPS).

### DETAILED DESCRIPTION OF THE INVENTION

Inflammation used herein is the biological response of vascular tissues to harmful stimuli, such as pathogens, damaged cells, or irritants. The disorders associated with inflammation include but are not limited to asthma, autoimmune diseases, Rheumatoid arthritis, hypersensitivities, and the like. Leukocytes play a major role in inflammatory and also release inflammatory mediators which develop and maintain the inflammatory response, such as IFN-γ, IL-8, IL-1, TNF-α, and so on.

Any disease that results from such an aberrant immune response is termed an autoimmune disease. Several mechanisms are thought to be operative in the pathogenesis of autoimmune diseases, against a backdrop of genetic predisposition and environmental modulation. Cytokine dysregulation which may induce severe inflammation is also a commonly mechanism resulting in autoimmune disease.

Cytokines are a category of signal protein and glycoprotein, which are critical to the development and functioning of both the innate and adaptive immune response. They are often secreted by immune cells while have encountered a pathogen, thereby activating and recruiting further immune cells to increase the system's response to the pathogen.

Interleukins are a group of cytokines that were first seen to be expressed by white blood cells as a means of communication. Interleukin-6 (IL-6) secreted by macrophage, TH2-cells, B cells, astrocytes or endothelium targets T cells and induces acute phase reaction, hematopoiesis, differentiation or inflammation. Interleukin-1β (IL-1β) also associated with inflammation is produced by macrophages, monocytes and dendritic cells.

The current view of the cytokine network in rheumatoid joints supports the notion that TNF-α activates a cytokine cascade characterized by the simultaneous production of pro-inflammatory cytokines such as IL-1 and IL-6. Cytokines such as TNF-α, IL-1, and IL-6 have been identified as key molecules in the pathogenesis of RA. In the present invention, the effects of Glycine tomentella Hayata on cytokines production are described (especially IL-6).

The present invention provides a method for inhibiting cytokine expression of a patient comprising administering the patient an effective amount of organic extract from Glycine tomentella Hayata, wherein the cytokine is selected from the group consisting of interleukin-1β (IL-1β), interleukin-6 (IL-6), transgluaminase type 2 (TG2) and matrix metalloproteinase 9 (MMP-9). The organic extract is extracted by ethanol and the organic extract further comprises a pharmaceutically acceptable carrier.

In the embodiment of the present invention, the cytokine is interleukin-1β interleukin-6, interleukin-8, interleukin-15, TG2, MMP-9, TGF-β, TNF-α or FGF. Especially, interleukin-1β, interleukin-6, TG2 or MMP-9 is preferred.

The Glycine tomentella Hayata used herein is prepared from the root of Glycine tomentella Hayata and extracted by 95% ethanol.

The method used for inhibiting cytokines, TG2 or MMP-9 further comprises administering pharmaceutically acceptable additives, carriers, diluents, excipients and/or fillers may be present in the method. The formulations can be prepared in any desirable medicine dosage forms, such as a lozenge, tablet, film coated tablet, capsule, soft capsule, granule, powder, pill, solution, emulsion, injection solution, injection, ointment, cream, spray, inhalant, soft gel, liquid, honey ball, lotion for oral, injection, topical, transmucosal or transdermal administrations.

The term “pharmaceutically acceptable carrier” used herein means any carrier that is pharmaceutically acceptable and has the desired pharmacological properties. Such carriers include salts that may be derived from an inorganic or organic acid, or an inorganic or organic base, including amino acids, which is not toxic or undesirable in anyway. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane and arene-sulfonic acids such as methanesulfonic acid, benzenesulfonic acid, sulfonic acid, and phosphatic acid). When there are two acidic groups present, a pharmaceutically acceptable salt may be a mono-acid-mono-salt or a di-salt; and similarly, where there are more than two acidic groups present, some or all of such groups can be salified

The examples below are non-limiting and are merely representative of various aspects and features of the present invention.

### EXAMPLES

### Example 1

### Extract of I-Tiao-Gung

Glycine tomentella Hayata was obtained from the Kinmen Doctor Wang I-Tiao-Gung limited company in the Kinmen Islands and authenticated by matching it with a specimen from Professor Hsien-Cheh Chang in Taiwan China Medical College.

The dry root of Glycine tomentella Hayata (50 g) was grounded and extracted with 95% ethanol (500 ml) at a ratio of 1:10 (wt/vol) and refluxed for 2 h at 75° C. twice. After evaporation of organic solvent under reduced pressure followed by lyophilization (32.8° C.), 3.7558 dry powder was obtained.

### Example 2

### Effect of I-Tiao-Gung on Cell Viability

The cytotoxicity of I-Tiao-Gung was determined by exposing RAW264.7 cell (Bioresource Collection and Research Center, Taiwan). RAW264.7 cells were cultured in DMEM containing 10% fetal bovine serum (Biological Industeries), 2 mM glutamine, 1 mM pyruvate, 1% non-essential amino acid, 1000 U/ml penicillin, 0.0025 mg/ml amphostericin, and 1 mg/ml streptomycin (Biological Industries). Cells were maintained in a humidified incubator at 37° C. in 5% CO₂.

Cell viability was quantitatively estimated by MTT assay, where the mitochondrial-dependent reduction of 3-(4,5-cimethylthiazol-2-yl)-2, 5-diphenyl tetrazolium bromide (MTT) to purple formazan. Cells were incubated with MTT (10%) for 4 h at 37° C. in 5% CO₂. Then, the medium was removed, and formazan crystals were dissolved in DMSO. The converted dye was quantitated by measurement of the absorbance at 570 nm. Cell viability after 24 h of I-Tiao-Gung treatment was greater than 95% when compared with control. NO differences was found in group up to I-Tiao-Gung 500 μg.

### Example 3

### Effects of I-Tiao-Gung on Proinflammatory Cytokine Release

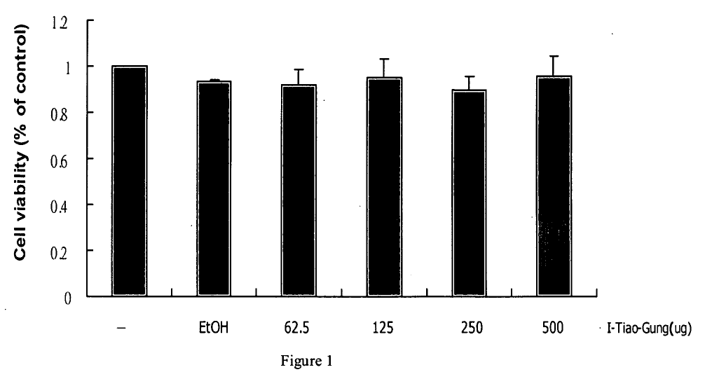

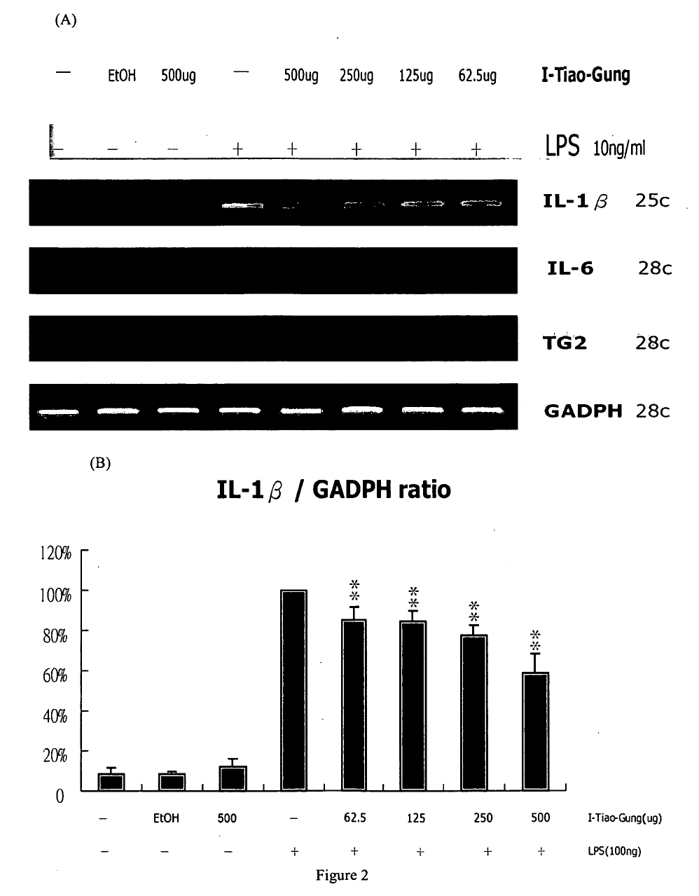

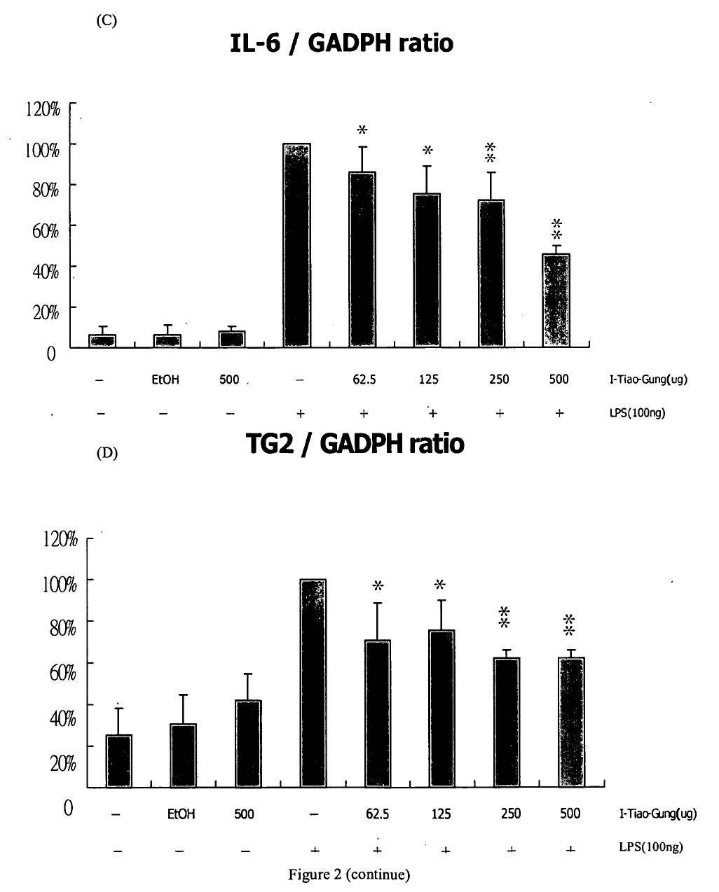

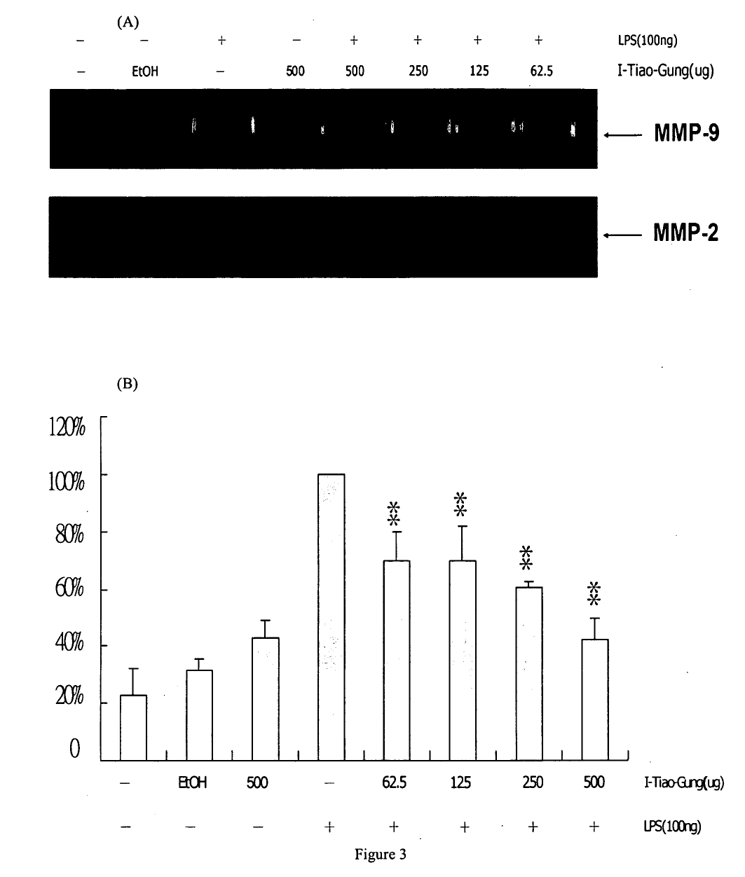

The present invention investigated the therapeutic properties of the ethanol extract of I-Tiao-Gung, the invention choose mouse macrophage RAW264.7 cell lines to characterize changes in inflammatory responses. To determine whether I-Tiao-Gung decrease LPS-induced IL-1β, IL-6, TG2 mRNA, RAW264.7 cells were incubated 24 h with the indicated concentrations of I-Tiao-Gung and then incubated for 2 h with 10 ng/ml LPS (FIG. 2). The invention found IL-1β, IL-6 and TG2 mRNA stimulated by LPS was inhibited by I-Tiao-Gung in a dose-dependent manner. Compared with FIG. 1, these results implied that the inhibition of IL-1β, IL-6 and TG2 mRNA synthesis was not due to cell death.

Total RNA was isolated from RAW264.7 cells by using the Trizol reagent protocol (Sigma). Two micrograms of total RNA was denatured at 65° C. with 1 μl oligo-dT (Promega C110A) prime, 4 μl dNTPs (10 mM) for 5 min in 12 ml final volume. Primers-RNA mixes were cooled on ice, 1 μl M-MLV reverse transcriptase (Invitrogen 28025-013), 1 μl RNase inhibitor (Promega) and 4 μl 5× RT-buffer were added in 20 ml final volume. PCRs were performed at the following conditions: 94° C. for 5 min, annealing at 54° C. for 1 mina), and DNA synthesis at 72° C. for 2 min, followed by 28 cycles. Sequences for the PCR primers follows: 5′-TCCATGAGCTTTGTACAAGGA-3′, 5′-AGCCCCATACTTTAGGAAGACA-3′ (forward and reverse mouse IL-1β probe), 5′-GTTCTCTGGGGTGGA-3′, 5′-TGTACTCCAGGTAGCTA-3′ (forward and reverse mouse IL-6 probe), 5′-TGATGACCGGGAGGACATCA-3′, 5′-GATTCCCAGGTAGAGATCTC-3′ (forward and reverse mouse TG2 probe) 5′-TCACTCAAGATTGTCAGCAA-3′, 5′-GATCCACGACGGACACATT-3′ (forward and reverse mouse GAPDH probe). The amplified PCR products were subjected to electrophoresis on a 2% agarose gel.

### Example 4

### Effects of I-Tiao-Gung on Matrix Metalloproteinase

Metalloproteinase-9 (MMP-9), also termed gelatinase B, in sera and synovial fluid (SF) from patients with RA. Marked inflammation was found in the limb joints. Both IL-1β and MMP-9 expression played a central role in the inflammatory reaction in acute stage. The present invention Gelatin-zymography was used to determine whether I-Tiao-Gung have any effect on MMP-9 activity. To detect MMP-9 activity, cell culture media were collected and concentrated. Electrophoresis was performed on zymogram gelatin gels (SIGMA). After the required developing time, gels were stained with Coomasie Blue (USBiological). Images were obtained with an Alpha-Imager2200. Significantly, expression of MMP-9 was reduced by I-Tiao-Gung treatment in a dose-dependent manner compared with cells stimulated only by LPS.

One skilled in the art readily appreciates that the present invention is well adapted to carry out the objects and obtain the ends and advantages mentioned, as well as those inherent therein. The cell lines, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for inhibiting cytokine expression of a patient comprising administering the patient an effective amount of organic extract from Glycine tomentella Hayata, wherein the cytokine is selected from the group consisting of interleukin-1β (IL-1β), interleukin-6 (IL-6), transgluaminase type 2 (TG2) and matrix metalloproteinase 9 (MMP-9).

2. The method of claim 1, wherein the Glycine tomentella Hayata is prepared from the root of Glycine tomentella Hayata.

3. The method of claim 1, wherein the organic extract is extracted by ethanol.

4. The method of claim 3, wherein the organic extract further comprises a pharmaceutically acceptable carrier.

5. The method of claim 1, wherein the patient suffers from Rheumatoid arthritis.


SEQUENCE LISTING (8 sequences)

SEQ ID NO: 1 - DNA, 21 bp, Artificial
  mouse IL-1beta]-F
tccatgagctttgtacaagga

SEQ ID NO: 2 - DNA, 22 bp, Artificial
  mouse IL-1beta]-R
agccccatactttaggaagaca

SEQ ID NO: 3 - DNA, 15 bp, Artificial
  mouse IL-6-F
gttctctggggtgga

SEQ ID NO: 4 - DNA, 17 bp, Artificial
  mouse IL-6-R
tgtactccaggtagcta

SEQ ID NO: 5 - DNA, 20 bp, Artificial
  mouse TG2-F
tgatgaccgggaggacatca

SEQ ID NO: 6 - DNA, 20 bp, Artificial
  mouse TG2-R
gattcccaggtagagatctc

SEQ ID NO: 7 - DNA, 20 bp, Artificial
  mouse GAPDH-F
tcactcaagattgtcagcaa

SEQ ID NO: 8 - DNA, 19 bp, Artificial
  mouse GAPDH-R
gatccacgacggacacatt